(19) United States
(12) Patent Application Publication
(10) Pub. No.: US 2021/0052142 A1
(43) Pub. Date: Feb. 25, 2021

(54) METHODS AND APPARATUS FOR CABLE ATTACHMENT SYSTEM

(71) Applicant: High Speed Interconnects, LLC, Scottsdale, AZ (US)

(72) Inventors: Antonio De La Rosa, Scottsdale, AZ (US); Todd Albertson, Portland, OR (US)

(21) Appl. No.: 17/000,333

(22) Filed: Aug. 23, 2020

Related U.S. Application Data
(60) Provisional application No. 62/890,770, filed on Aug. 23, 2019.

(51) Int. Cl. A61B 1/00; A61M 39/08; A61M 39/10; A61B 1/05; A61B 1/045

(52) U.S. Cl. CPC A61B 1/00114; A61M 39/08; A61M 39/10; A61B 1/05; A61B 1/2736; A61B 1/00124; A61B 1/0011; A61B 1/045; A61B 1/00066

(57) ABSTRACT

A cable attachment system and method for use with micro sensing component in a sterile environment. The cable attachment system may comprise a sensor, a cable connected to the sensor at a first end, a connector coupled to a second end of the cable, and a board coupled to the connector. A medical grade tubing may encase the connection of the sensor to the cable and a medical grade housing may encase the connection of the board to the connector and connected to the tubing. The medical grade housing and tubing are configured to create a sealed internal volume in the sterile environment.

### CROSS-REFERENCE TO RELATED APPLICATIONS

The present application claims the benefit of U.S. Provisional Application Ser. No. 62/890,770, filed Aug. 23, 2019, entitled “Methods and Apparatus for Cable Attachment System” and incorporates the disclosure of the application by reference.

### BACKGROUND OF THE TECHNOLOGY

Micro sensing components such as endoscopes, guidewires, catheters, cameras, and the like require small form factors that inherently create installation and manufacturing concerns for discrete cable or coaxial cable termination. For example, devices such as endoscopes, guidewires, catheters, or cameras typically have a cable assembly to facilitate the signal transfers to or from the device and/or to provide power. However, because these types of devices have size constraints on the order of 3 millimeters or less, cable design and proper termination of the cable at the proximal/system end can be problematic once the assembly is terminated with a connector.

Common methods of connecting cables to processing units for the sensors and power sources use soldering, connectors and the like. These types of connections may create a manufacturing concern since connecting such a small wire requires precision slowing down the manufacturing process and potentially leading to excessive manufacturing efficiencies or assembly quality problems. Furthermore, this type of connection increases the chance for interference, which can cause any number of issues.

Other issues may arise when the environment where the cable assembly is used is subject to higher standards of cleanliness, such as a clean room for certain types of laboratories used in scientific research, including the manufacture of pharmaceutical items, microprocessors, medical devices, and the like.

### SUMMARY OF THE TECHNOLOGY

Methods and apparatus for a cable attachment system according to various aspects of the present technology include a sensor, a cable, and a connector. The cable is coupled between the sensor and connector. The cable attachment system may comprise tubing with the cable extending within the tubing. The cable attachment system may comprise a board, which is coupled to the connector. The cable attachment system may also comprise an external housing and a hand piece.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 representatively illustrates a side view of a sensory assembly component having a sensor, cable, and connector in accordance with an exemplary embodiment of the present technology;

FIG. 2 representatively illustrates a side view of the sensory assembly component of FIG. 1 and further inserted in tubing in accordance with an exemplary embodiment of the present technology;

FIG. 3 representatively illustrates a side view of the sensory assembly component of FIG. 2 with the connector inserted into a connector on a board in accordance with an exemplary embodiment of the present technology;

FIG. 4 representatively illustrates a side view of the sensory assembly component of FIG. 3 with the connector inserted into a connector and epoxied on a board in accordance with an exemplary embodiment of the present technology;

FIG. 5 representatively illustrates a side view of the sensory assembly component of FIG. 3 with a housing cover placed around the connectors on the board in accordance with an exemplary embodiment of the present technology;

FIG. 6 representatively illustrates a side view of the sensory assembly component of FIG. 5 with tube extended from the housing cover to the sensor in accordance with an exemplary embodiment of the present technology;

FIG. 7 representatively illustrates a partial, side view of the sensory assembly component of FIG. 6 with the tube and cable extending through a hand piece in accordance with an exemplary embodiment of the present technology;

FIG. 8A representatively illustrates a perspective view of the sensory assembly component with the tube and cable extending through a hand piece in accordance with an exemplary embodiment of the present technology;

FIG. 8B representatively illustrates a side view of the sensory assembly component with the tube and cable extending through a hand piece in accordance with an exemplary embodiment of the present technology;

FIG. 8C representatively illustrates a top view of the sensory assembly component with the tube and cable extending through a hand piece in accordance with an exemplary embodiment of the present technology; and

FIG. 8D representatively illustrates a side, cross-section view of the sensory assembly component with the tube and cable extending through a hand piece in accordance with an exemplary embodiment of the present technology.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present technology.

### DETAILED DESCRIPTION OF EXEMPLARY EMBODIMENTS

The present technology may be described in terms of functional block components and various processing steps. Such functional blocks may be realized by any number of hardware or software components configured to perform the specified functions and achieve the various results. For example, the present technology may employ various cables, sensors, dielectrics, connection types, circuit cards/boards, and the like, which may carry out a variety of analog or digital functions, such as MIPI, I2C, LVDS or any other suitable configuration. The signals may comprise any suitable electrical signals, for example a combination of data, control signals, and power. In addition, the present technology may be practiced in conjunction with any number of applications, and the system described is merely one exemplary application for the technology. Further, the present technology may employ any number of conventional techniques for providing analog or digital control signals, reducing noise, cross-talk, attenuation, impedance, controlling power, and the like.

Methods and apparatus for a cable attachment system according to various aspects of the present technology may operate in conjunction with any suitable electronic sensor, video system, data collection system, and/or other electronic device. Various representative implementations of the present technology may be applied to any appropriate system for imaging such as a temporarily insertable camera system like an endoscope or any other suitable technology.

Cable attachment systems may be used in various applications where flexibility, space savings, or production constraints limit the applicability of rigid circuit boards or hard wiring. Cable attachment systems according to various embodiments may be suited for use in various environments, such as, a cleanroom or operating room where it is critical that the components are not subject to various environmental particulates, such as dust, airborne organisms, and the like. A cleanroom is typically used with types of laboratories used in scientific research, including the manufacture of pharmaceutical items, microprocessors, medical devices, and the like. A cleanroom or related facility may also be used in hospital settings where cleanliness of surgical instruments, such as endoscopes, catheters, guidewires, or a flexible introducer sheath is required. As is understood, endoscopes are typically inserted in a human body to look inside the body. For example, some types of endoscopes may include but are not limited to gastroscopes, bronchoscopes, colonoscopes, laryngoscopes, cystoscopes, duodenoscopes, enteroscopes, ureteroscopes, hysteroscopes. An endoscopy procedure uses an endoscope typically to examine interior organs or cavities within the body. Catheters are typically thin tubes made from various medical grade materials and serve a broad range of functions. Catheters are typically medical devices that may be inserted within a body, duct, vein, vessel or cavity to treat diseases or perform surgical procedures. Catheters may also be used as drains, to administer fluids and gasses or as access for surgical instruments. Based on areas where catheters and endoscopes are used it is important that the materials and components used therewith are of optimal grade and cleanliness.

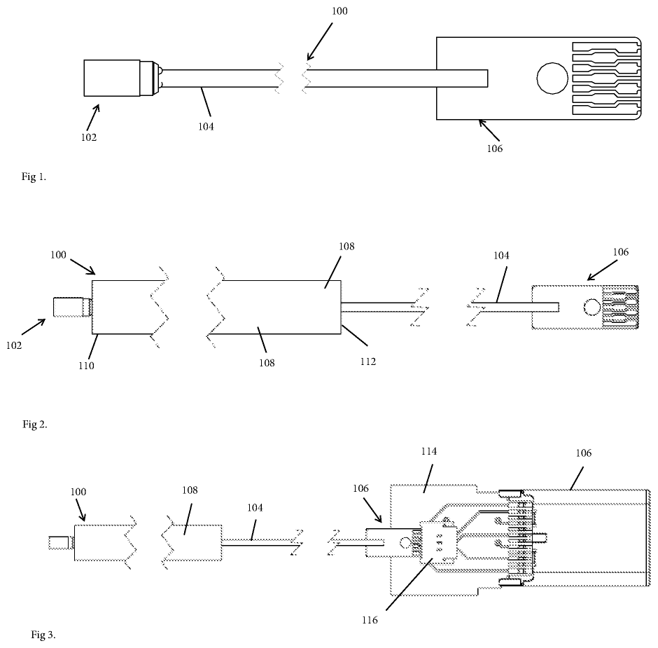

Referring now to FIGS. 1-7 a cable attachment system 100 according to various aspects of the present technology may be used within micro sensing components such as endoscopes, guidewires, catheters, cameras, and the like in a clean room environment with medical grade components. According to various embodiments and shown in FIG. 1, the cable attachment system 100 may comprise a sensor 102, a cable 104, and a connector 106. The sensor 102 may be coupled to the cable 104 by any known method. In one embodiment the sensor 102 may be mechanically coupled to the cable by solder. In other embodiments, the sensor 102 may be coupled to the cable 104 by conductive epoxy, mechanical methods, welded, and the like. The cable 104 connects the sensor to the connector 106. The connector 106 may be coupled to the cable 104 by any known method. In one embodiment the connector 106 may be mechanically coupled to the cable 104 by solder. In other embodiments, the connector 106 may be coupled to the cable 104 by conductive epoxy, mechanical methods, welded, and the like.

In various embodiments, the sensor 102 may comprise any suitable component including a camera, a complementary metal-oxide semiconductor (“CMOS”) sensor, charged-coupled device (“CCD”) sensors with or without a lens, an ultrasound transducer, a pressure sensor, an LED or a plurality of LEDs, and the like.

In various embodiments, the connector 106 may comprise a flex circuit or a rigid PCB, USB, HDMI, and the like. The flex circuit, shown in FIG. 1 may comprise a width of approximately 1 mm-6 mm. In one embodiment, the flex circuit may comprise a width of 2 mm-2.5 mm.

In various embodiments the cable 104 may comprise coaxial cables. In general, coaxial cables may be used to transmit signals from a source device at a source end to a receiving end such as a display or memory device. The signals may comprise any suitable electrical signals, for example a combination of data, control signals, and power. For example, in one embodiment for use with sensor 102 comprising an imaging system using a camera, the cable 104 may provide electrical power to the camera. The camera may be configured with an integrated circuit such as a CMOS imaging sensor, an array, a ball grid array (“BGA”), or other sensing device that is connected directly to the cable 104. A second coaxial cable may transmit a clock signal between the camera and the connector 106 of the cable attachment system 100. A third coaxial cable may transmit an image signal from the camera to the receiving end where the image signal may be displayed or analyzed for processing. In alternative embodiments, additional coaxial cables may be included to transmit additional power lines or signals as required. Similarly, fewer cables may be used if the particular application requires the transmission of fewer signals or less power lines through the cable attachment system 100.

The coaxial cables may comprise any suitable type of coaxial cable, such as a cable comprising a center conductor, a dielectric surrounding the center conductor, and a shield covering the dielectric.

Referring now to FIG. 2, in various embodiments, the cable attachment system 100 may comprise tubing 108 made from medical grade materials. Some examples include polymers, especially silicone, nylon, polyethylene terephthalate (PET), polyurethane, fluoropolymers, polyvinyl chloride (PVC), TBE, thermoplastic vulcanizates (TPV), and the like. In various embodiments, the tubing 108 may comprise an outer diameter of 2 mm-8 mm millimeters and an inner diameter of 1.5 mm-6 mm. In use, the diameter of the connector 106 must be less than the dimeter of the tubing 108 such that the connector 106 may be threaded through the tubing 108.

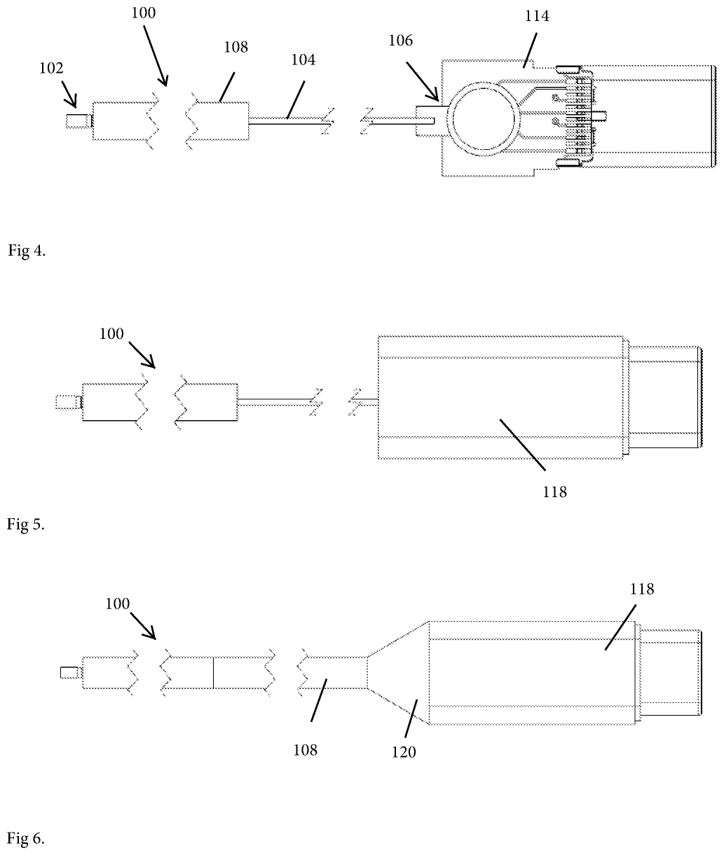

To assemble the cable attachment system 100 of FIG. 2, the connector 106 is inserted within a first end 110 of the tubing 108 and the connector 106 is threaded through the tubing 108 out of a second end 112 such that the connector 106 is exposed. The connector 106 may be pushed or pulled through the tubing 108. In one embodiment the connector 106 may be pulled through with a wire or flexible filament/thread (not shown). The wire or flexible filament/thread may be attached to the connector 106, inserted into the first end 110, threaded through the tubing 108 to the second end 112. The wire or flexible filament/thread may then be used to pull the connector 106 from the first end 110 to the second end 112 of the tubing. The connector 106 may then be coupled to a board 114, as shown in FIG. 3. In one embodiment, the connector 106 comprising a flex circuit or rigid printed circuit board “PCB” circuit may be coupled to a receptacle 116 on the board 114. In one embodiment the connector 106 may be mechanically coupled to the board 114, by clamping, welding, soldering, epoxy and the like. In one embodiment, shown in FIG. 4, the connector 106 comprising a flex circuit or rigid PCB circuit may be coupled to a receptacle 116 on the board 114 and then epoxy may be used to permanently couple the connector to the board 114. The user may install a housing 118 around the board 114.

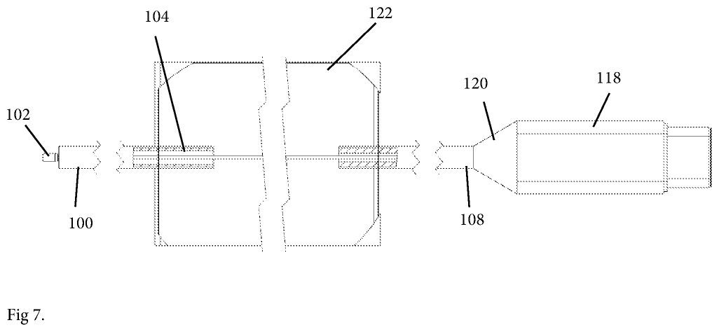

Referring now to FIG. 5, the cable attachment system 100 of FIG. 3 or 4 may comprise the housing 118, used to cover the board 114. The material for the housing 118 may comprise any suitable medical grade material including rigid plastic, flexible plastic. Some examples include but are not limited to polyurethane, PVC, thermoplastic elastomer (TPE), TPVs for a flexible housing and thermoplastic resin such as ABS, amorphous engineering material, such as PC, polyphenylsulfone (PPSU), PVC, semi crystalline thermal plastic, such as PP, and nylon for a rigid housing. As shown in FIG. 6, the housing 118 and tubing 108 may be extended and connected such that cable 104 and connector 106 are completely contained therein. In one embodiment the housing 118 and tubing 108 may be connected by a transition piece 120.

Referring now to FIG. 7, in various embodiments, the cable attachment system 100 may comprise a hand piece 122, used to control the sensor 102. The hand piece 122 could be any number of products that may be used to control the sensor 102. In various embodiments, the hand piece 122 may be used to manually steer the sensor 102 (e.g. tip of a flexible endoscope) to navigate through any path in the human body. In various embodiments, a physician may hold the hand piece to steer the sensor 102. In one embodiment, the hand piece 122 may be used to manually steer the sensor 102 (e.g. tip of a flexible endoscope) to navigate through an endoluminal path. In one embodiment, the hand piece 122 may be used to manually steer the sensor 102 (e.g. tip of a flexible endoscope) in as gastroscopy, to navigate the esophagus and the stomach via the mouth. In one embodiment, the hand piece 122 may be used to manually steer the sensor 102 (e.g. tip of a flexible endoscope) in a colonoscopy which involves the inspection of the colon via the rectum.

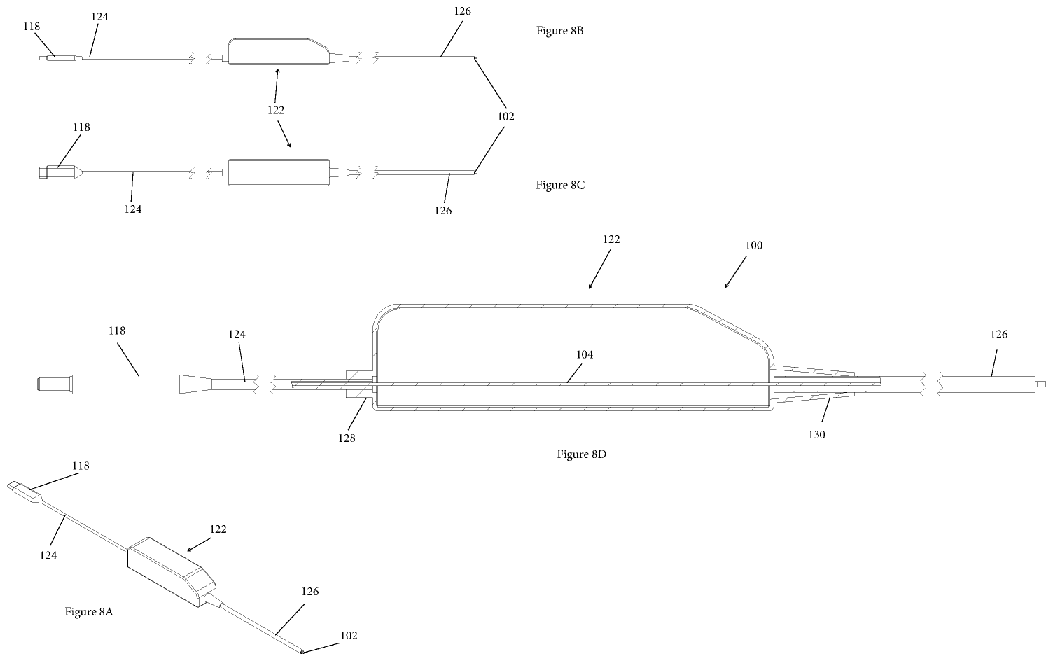

Referring now to FIGS. 8A-D, in various embodiments, the cable attachment system 100 may comprise the hand piece 122, used to control the sensor 102. The hand piece 122 could be any number of products that may be used to control the sensor 102. The tubing 108 may comprise a proximal tubing 124 and a distal tubing 126. The proximal tubing 124 extends from a first end of the hand piece 122 to the housing 118 (containing the connector 106 and board 114) while the distal tubing 126 extends from a second end of the hand piece 122 to the sensor 102. The proximal tubing 124 may be received within a connection port 128 in the first end of the hand piece 122 while distal tubing may be received within a connection port 130 in the second end of the hand piece 122. The proximal and distal tubing 124, 126 may be secured within the connection ports 128, 130 by any known method. In one embodiment the proximal and distal tubing 124, 126 may be secured within the connection ports 128, 130 mechanically. In one embodiment, the proximal and distal tubing 124, 126 may be glued in a strain relief to the connection ports 128, 130. The cable 104 extends within the housing as shown in FIG. 8 D

Current systems require an end user to put the cable through the tubing without the connector (flex circuit) installed. The flex circuit must then be installed after the cable is threaded through the tubing. The present application allows for the connector 106 (flex circuit) to be threaded through the tubing 108 in a sterile facility or clean room. The connector 106 and tubing 108 can then be continued through additional components (hand pieces 122, proximal jackets, housings, etc.) and connected to the receptacle 116 on the board 114. The board 114 has a connector (not shown) on the other side that is a standard off the shelf connector (USB, HDMI, etc.) which can then connect to virtually any other component as desired.

Thus, the cable attachment system 100 described above provides a low cost solution, where no additional components exist between the camera/sensor 102 and the connector 106, and the customer/end user does not have to do any soldering after the assembly process to couple any connector to the cable.

The cable attachment system 100 described above may be used in surgical instruments, such as endoscopes, catheters in a surgical environment where the components should not be subjected to various environmental particulates, such as dust, airborne organisms, and the like. As discussed above a cleanroom is typically used with types of laboratories used in scientific research, including the manufacture of pharmaceutical items, microprocessors, medical devices, and the like. A cleanroom or related facility may also be used in hospital settings where cleanliness of surgical instruments, such as endoscopes, catheters, is required. The cable attachment system 100 described above would be suitable for such an environment since the sensor 102, cable 104, tubing 108, connector 106, and optionally the board 114 may be assembled in a sterile environment, such as a cleanroom. The sensor 102, cable 104, connector 106, and optionally the board 114 may be completely sealed within the combination of the tubing 108 and housing 118. This sealed nature provides a sterile system such that the components are not exposed to harmful environmental elements. Thus, the end user or doctor may simply open the packaging, which contains the fully contained or enclosed cable attachment system 100 and it could immediately be used on the patient.

These and other embodiments for methods of forming the cable attachment system 100 may incorporate concepts, embodiments, and configurations as described above. The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The technology has been described with reference to specific exemplary embodiments. Various modifications and changes, however, may be made without departing from the scope of the present technology. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any order, unless otherwise expressly specified, and are not limited to the explicit order presented in the specific examples. Additionally, the components and/or elements recited in any apparatus embodiment may be assembled or otherwise operationally configured in a variety of permutations to produce substantially the same result as the present technology and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises,” “comprising,” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology.

1. A cable attachment system for use with micro sensing component in a sterile environment, the cable attachment system comprising:
a sensor;
a cable connected to the sensor at a first end;
a connector coupled to a second end of the cable;
a medical grade tubing encasing the connection of the sensor to the cable, wherein a diameter of the tubing is greater than a diameter of the connector;
a board coupled to the connector;
a medical grade housing encasing the connection of the board to the connector and connected to the tubing, wherein the housing and tubing are configured to create a sealed internal volume in the sterile environment.

2. The cable attachment system of claim 1, wherein the sensor comprises one of a camera, a CMOS sensor, a CCD Sensor, an ultrasound transducer, a pressure sensor, or an LED.

3. The cable attachment system of claim 1, wherein the connector comprises one of a flex circuit or rigid PCB.

4. The cable attachment system of claim 3, wherein the flex circuit comprises a width of approximately 1 mm to 6 mm.

5. The cable attachment system claim of claim 3, wherein the flex circuit comprises a width of approximately 2 mm to 2.5 mm.

6. The cable attachment system of claim 1, wherein the medical grade tubing comprises an outer diameter of approximately 2 mm to 8 mm and an inner diameter of approximately 1.5 mm to 6 mm.

7. The cable attachment system of claim 1, further comprising a hand piece configured to control the sensor, wherein the cable extends through the hand piece.

8. The cable attachment system of claim 7, wherein the medical grade tubing comprises a proximal end coupled to a first end of the hand piece and a distal end coupled to a second end of the housing.

9. The cable attachment system of claim 8, wherein the proximal end of the medical grade tubing is received in a connection port in the first end of the hand piece and distal end of the tubing is received in a connection port in the second end of the hand piece.

10. A method of assembling the cable attachment system of claim 1 comprising:
inserting the connector within a first end of the medical grade tubing;
receiving the connector from a second end of the medical grade tubing;
coupling the connector to a receptacle on a board;
installing the medical housing over the board; and
connecting the medical grade tubing to the medical grade housing to create a sealed internal volume.

11. The method of claim 10, wherein the connector is manually pushed through the medical grade tubing.

12. The method of claim 10, wherein a wire is coupled to the connector, inserted within the medical grade tubing, and configured to pull the connector through the medical grade tubing.

13. The method of claim 10, wherein the connector comprises a flex circuit.

14. The method on claim 13, further comprising inserting the connector through a hand piece configured to control the sensor.

15. A method of assembling a cable attachment system for use with micro sensing component in a sterile environment comprising a sensor, a cable coupled to the sensor, and a connector coupled to the cable, the method comprising:
inserting the connector within a first end of a medical grade tubing;
receiving the connector from a second end of the medical grade tubing;
coupling the connector to a receptacle on a board;
installing a medical grade housing over the board; and
connecting the medical grade tubing to the medical grade housing to create a sealed internal volume.

16. The method of claim 15, wherein the connector is pushed through the medical grade tubing.

17. The method of claim 15, wherein the connector is pulled through the medical grade tubing with a wire.

18. The method of claim 17, wherein the connector comprises a flex circuit.

19. The method of claim 18, further comprising inserting the connector through a hand piece configured to control the sensor.

20. The method of claim 15, wherein the connector comprises one of a flex circuit or rigid PCB.

21. The method of claim 20, wherein the flex circuit comprises a width of approximately 1 mm to 6 mm.

22. The method of claim 20, wherein the flex circuit comprises a width of approximately 2 mm to 2.5 mm.

23. The method of claim 15, wherein the medical grade tubing comprises an outer diameter of approximately 2 mm to 8 mm and an inner diameter of approximately 1.5 mm to 6 mm

24. The method of claim 15, further comprising inserting the cable into a hand piece configured to control the sensor.

25. The method of claim 24, wherein the medical grade tubing comprises a proximal end coupled to a first end of the hand piece and a distal end coupled to a second end of the housing.

26. The method of claim 25, wherein the proximal end of the medical grade tubing is received in a connection port in the first end of the hand piece and distal end of the tubing is received in a connection port in the second end of the hand piece.
